(19) United States
(12) Patent Application Publication
(10) Pub. No.: US 2009/0124850 A1
(43) Pub. Date: May 14, 2009

(54) Portable player for facilitating customized sound therapy for tinnitus management

(76) Inventors: F. Richard Moore, San Diego, CA (US); Erik Viirre, San Diego, CA (US)

Correspondence Address: LIU & LIU, 444 S. FLOWER STREET SUITE 1750, LOS ANGELES, CA, 90071, US

(21) Appl. No.: 12/290,640

(22) Filed: Oct. 30, 2008

Related U.S. Application Data
(63) Continuation-in-part of application No. 12/215,385, filed on Jun. 25, 2008.
Application No. 12/215,385 is a continuation-in-part of application No. 12/286,895, filed on Oct. 1, 2008.
(60) Provisional application No. 61/001,209, filed on Oct. 30, 2007.

(51) Int. Cl. A61M 21/00; G06F 17/00

(52) U.S. Cl. 600/28; 700/94

(57) ABSTRACT

A portable player for facilitating implementation of customized sound therapy to a patient. The PSP is designed to producing sounds at fairly low listening levels, wherein in order for effective habituation to occur. The PSP generates frequencies covering the entire audible range (about 20-20,000 Hz) at low distortion. The PSP is operatively coupled to a sound matching station (SMS) via a suitable interface (e.g., a USB interface), is connected, wired or wirelessly, to audio output transducers (e.g., headphones) for both the patient and the CST operator during the matching session, then detached for patient's use during habituation therapy. The PSP records automatically the patient's usage, by building a record of the date and time when the PSP was turned on and off, and the sound volume used at that time. This information can be downloaded at the next visit onto the SMS and reviewed and analyzed for further treatments.

This application claims the priority of Provisional Patent Application No. 61/001,209, filed Oct. 30, 2007; this application is a continuation-in-part of U.S. patent application Ser. No. 12/215,385, filed Jun. 25, 2008, and this application is a continuation-in-part application of U.S. patent application Ser. No. 12/286,895, filed Oct. 1, 2008, which application is a continuation-in-part of U.S. patent application Ser. No. 12/215,385. These earlier applications and all patent documents and other publications disclosed herein below are fully incorporated by reference, as if fully set forth herein.

### BACKGROUND OF THE INVENTION

1. Field of the Invention

The present invention generally relates to medical apparatus, in particular systems and methods for tinnitus therapy, and more particularly to a portable player for application of Customized Sound Therapy (CST) for tinnitus relief and management.

2. Description of Related Art

Tinnitus is a debilitating condition defined as the sensation of “ringing in the ears” in the absence of external stimuli. The American Tinnitus Association reports that approximately 36 million Americans have some form of tinnitus, with over 12 million Americans suffering from tinnitus so severe that quality of life is seriously compromised. The United States Veterans Administration alone spends over $500 million annually on tinnitus related disability benefits for former U.S. Armed Forces personnel. Over one-third of Americans over the age of 65 are affected by tinnitus, and thus, it is also the tenth most common presenting complaint among the elderly in primary care. Given the aging demographics of the U.S., the prevalence of this condition is only expected to rise in coming years.

For many years, investigators have focused on permanent changes in the peripheral auditory system as being primarily responsible for the etiology of tinnitus. However, recent research suggests that while tinnitus may be triggered by events in the periphery, the mechanisms that transform tinnitus into a persistent and debilitating condition are located in the central nervous system. This implies that therapy should be directed at central auditory function to treat tinnitus. Furthermore, tinnitus patients unintentionally condition themselves through negative reinforcement. It has been demonstrated, for instance, that cortical representation of tones associated with unpleasant sensations is enlarged [Gonzalez-Lima and Scheich, Neural Substrates for Tone-Conditioned Bradycardia Demonstrated With 2-Deoxyglucose. II. Auditory Cortex Plasticity. Behav. Brain Res. 1986; 20(3):281-93]. It has also been shown that neurons in the primary auditory cortex of gerbils change their response characteristics to tones coupled with aversive unconditioned stimuli [Ohl and Scheich, 1996]. A computational model of tinnitus based on animal studies shows that the limbic system is a necessary component for stabilizing the tinnitus perception. This model also explains how a peripheral hearing deficit leading to decreased auditory input can give rise to a specific tinnitus pitch.

U.S. Pat. No. 7,081,085 to Viirre et al., entitled “EEG feedback controlled sound therapy for tinnitus” teaches a method for treating tinnitus by habituation through use of neurological feedback, comprising the steps of connecting a subject through a set of attached headphones to an electronic sound. This patent, however, does not teach tinnitus treatment and management in the context of an integrated system to facilitate tinnitus therapy to be applied to patients more prevalently. Further, tinnitus treatment is conducted using relatively audio playback equipment, which are not efficient for applying therapy to individual patients.

U.S. patent application Ser. No. 12/215,385 (commonly assigned to the assignee of the present invention) discloses an integrated system developed for implementation of customized sound therapy (CST). The disclosed CST system includes apparatus, devices, components, processes and methods for treating, relieving, and managing tinnitus. The CST system is used by a person (e.g., a qualified healthcare professional, such as an otolaryngologist, an audiologist, or other qualified professional, or individual patients themselves with sufficient training) to test and treat a patient. One embodiment of the CST system comprises a system that includes: (1) a Sound Matching Station (SMS), which is a dedicated electronic device having a processing system including a CST application, an acoustic compiler for generating CST sounds, a graphical user interface user (GUI), an output for high-quality digital audio file output; (2) an audio device for playback of CST sounds to the patient. In one embodiment, the audio device includes a Portable Sound Player (PSP). The PSP using a stereophonic playback converts the digital audio file into CST sounds. The present application elaborates on the inventive aspects of such PSP.

### SUMMARY OF THE INVENTION

The present invention is directed to a portable player for facilitating implementing customized sound therapy (CST) to a patient.

The PSP is designed to producing sounds at fairly low listening levels, wherein in order for effective habituation to occur, the customized sound should be audible, but not loud enough to mask the tinnitus sound itself. The volume settings that tinnitus subjects use on their PSP give an estimate of the intensity of the tinnitus. The audio power involved is highly frequency-dependent, but in any case should not exceed the patient's threshold (e.g., approximately 80 dB SL (sensation level, i.e., dB above patient's threshold)). The PSP needs to be capable of generating frequencies covering the entire audible range (about 20-20,000 Hz) at low distortion.

The PSP is operatively coupled to a sound matching station (SMS) via a suitable interface (e.g., a USB interface), is connected, wired or wirelessly, to audio output transducers (e.g., headphones) for both the patient and the CST operator during the matching session, then detached for patient's use during habituation therapy. When the patient uses the PSP, it records automatically the patient's usage, i.e., it records (by building a record) the date and time when the PSP was turned on and off, and the sound volume used at that time. This information can be downloaded at the next visit onto the SMS and reviewed and analyzed for further treatments.

In a specific embodiment, the PSP includes the following structures, features and functions that are particularly desirable for CST for tinnitus management:
Continual, repeated playback of a single recorded sound of a preset duration (e.g., at least 5 minutes duration).
Stereophonic playback to allow for differential playback in each ear.
A balance control to allow the relative loudness in each ear to be adjusted.
Playback volume limitation limited to a certain volume level.
An internal date and time of day clock to allow internal logging of playback times and volumes.
Internal monitoring software to log playback dates, times, volumes, etc.
USB or other convenient interface to the SMS allowing exchange of sound and logging data, which should include playback times and volumes, and other patient data, such as ID and arbitrary text notes, such as sound specifications.
Transducers for one or both ears.
Provision for connection to multiple transducers in order to allow monitoring by SMS operator during sound customization or at other times.
Audio requirements: the sound playback should be at high quality. The analog audio output needs to be able to drive at least two sets of transducers with independent volume settings, allowing simultaneous sound monitoring by both the patient and the CST operator.

### BRIEF DESCRIPTION OF THE DRAWINGS

For a fuller understanding of the scope and nature of the invention, as well as the preferred mode of use, reference should be made to the following detailed description read in conjunction with the accompanying drawings. In the following drawings, like reference numerals designate like or similar parts throughout the drawings.

FIG. 1 illustrates a schematic block diagram of the CST system in accordance with one embodiment of the present invention.

FIG. 2 illustrates another schematic view of the CST system in accordance with one embodiment of the present invention.

FIG. 3 is a pictorial illustration of the patient's experience in connection with the CST system, in accordance with one embodiment of the present invention.

FIG. 4 is a screen display of the graphical user interface for the sound matching station, in accordance with one embodiment of the present invention.

FIG. 5 is a schematic diagram illustrating the functional components of the sound matching system, in accordance with one embodiment of the present invention.

FIG. 6 is a schematic flow diagram illustrating the operation of an audio device used in the CST system, in accordance with one embodiment of the present invention.

FIG. 7 is a perspective view of an audio device used in the CST system, illustrating its features, in accordance with one embodiment of the present invention.

FIGS. 8 and 9 are various additional views of the audio device in FIG. 7.

FIG. 10 illustrates Type I Noise: (a) waveform consisting of random values with linear interpolation between adjacent points; (b) The associated (normalized) right-half amplitude spectrum, in accordance with one embodiment of the present invention.

FIG. 11 illustrates Type I Noise: (a) spectrum from FIG. 10, by spectral convolution with a sinusoid, in accordance with one embodiment of the present invention; (b) spectrum similar to FIG. 10.

FIG. 12 illustrates frequency-dependent gain of a two-pole filter, in accordance with one embodiment of the present invention.

FIG. 13 is a schematic diagram of the components of the PSP, in accordance with one embodiment of the present invention.

FIG. 14 is a schematic block diagram of the components of the PSP, in accordance with one embodiment of the present invention.

FIG. 16 is a schematic block diagram of the uploading operation of the PSP.

FIG. 17 is a schematic block diagram of the downloading operation of the PSP.

FIG. 18 is a schematic block diagram of the audio output level change operation of the PSP.

FIG. 19 is a schematic block diagram of the logging operation of the PSP.

FIG. 20 is a schematic flow diagram of the firmware operations of the PSP.

FIG. 21 is a screen shot of the display at the SMS, which illustrates a folder structure of information uploaded to and downloaded from the SMS, in accordance with one embodiment of the present invention.

FIG. 22 is a screen shot of the display at the SMS, which further illustrates a folder structure of distributed provide information, including patient usage statistics.

### DETAILED DESCRIPTION OF EMBODIMENTS OF THE INVENTION

The present description is of the best presently contemplated mode of carrying out the invention. This description is made for the purpose of illustrating the general principles of the invention and should not be taken in a limiting sense. The scope of the invention is best determined by reference to the appended claims.

The detailed descriptions of the process of the present invention are presented in terms of schematics, functional components, methods or processes, symbolic or schematic representations of operations, functionalities and features of the invention. These descriptions and representations are the means used by those skilled in the art to most effectively convey the substance of their work to others skilled in the art. A software implemented method or process is here, and generally, conceived to be a self-consistent sequence of steps leading to a desired result. These steps require physical manipulations of physical quantities. Often, but not necessarily, these quantities take the form of electrical or magnetic signals capable of being stored, transferred, combined, compared, and otherwise manipulated.

Useful devices for performing the software implemented operations and functions of various aspects of the present invention include, but are not limited to, general or specific purpose digital processing and/or computing devices, which devices may be standalone devices or part of a larger system. These devices may be selectively activated or reconfigured by a program, routine and/or a sequence of instructions and/or logic stored in the devices. In short, use of the methods described and suggested herein is not limited to a particular processing configuration.

For purposes of illustrating the principles of the present invention and not by limitation, the present invention is described herein below by reference to the exemplary CST system developed by Tinnitus Otosound Products, Inc. However, it is understood that the present invention is equally applicable to systems of other configurations embodying the invention, without departing from the scope and spirit of the present invention.

CST consists of providing a patient with a recorded synthetic sound that matches as closely as possible his or her internal tinnitus sensation. This “customized sound” is created through interaction of the patient with a physician, audiologist, or other trained personnel (e.g., using a CST sound matching station (SMS)). In accordance with the present invention, once the matching sound is identified, it is replicated and made available to the patient via a player (e.g., a Portable Sound Player (PSP)). The patient carried the PSP from then on, listening to the customized sound at low audio levels for as many hours per day as is comfortable for the patient (a form of habituation therapy). During this time, the patient continually adjusts the playback volume of the customized sound to match the perceived level of his or her internal tinnitus sensation. It has been found that in the vast majority of cases, the audio level needed on the PSP to match the perceived level of the internal tinnitus sensation will go down over a period of days and weeks. This is the principle result of CST research and constitutes “calming” of the tinnitus sensation. By precisely replicating a patient's tinnitus experience with a custom generated frequency-specific sound delivered by, e.g., a portable digital sound device, and by selectively and continuously stimulating the same population of neurons affected by the tinnitus over a period of time with CST, corrective habituation occurs rapidly and efficiently.

Overview of CST System

The CST System includes apparatus, devices, components, processes and methods for treating, relieving, and managing tinnitus (one or more of these may be part of the tinnitus therapy applied to the patient). The CST System is used by a person (e.g., a qualified healthcare professional, such as an otolaryngologist, an audiologist, or other qualified professional, or individual patients themselves with sufficient training) to test, treat or provide therapy to a patient. One embodiment of the present invention comprises a system that includes: (1) a Sound Matching Station (SMS), which is a dedicated electronic device having a processing system including a CST application, an acoustic compiler for generating CST sounds, a graphical user interface (GUI), an output for high-quality digital audio file output; (2) an audio device for playback of CST sounds to the patient. In one embodiment, the audio device includes a Portable Sound Player (PSP). The PSP using a stereophonic playback converts the digital audio file into CST sounds, which can be heard through a pair of high fidelity earphones provided to the patient and one for the tester.

The target patient population for the CST System is adults (18 years and over, but age is not a limitation) who are presented with tinnitus, which may or may not be accompanied with hearing loss at the higher frequencies, and who are participating in a tinnitus management program. CST System consists of providing a patient with a recorded synthetic sound that matches as closely as possible the internal tinnitus sensation. This “customized sound” is created through interaction of the patient with a physician, audiologist, or other trained personnel, at a CST SMS. This enables a qualified healthcare professional to identify, with the patient's verbal input, the sounds that most closely match the patient's tinnitus. Once the matching sound is identified, it is available to the patient via a PSP. The patient carries the PSP from then on, listening to the customized sound, via high quality earphones, at low audio levels for as many hours per day as is comfortable for the patient (a form of habituation therapy). During this time, the patient continually adjusts the playback volume of the customized sound to match the perceived level of the internal tinnitus sensation. It has been found that in the vast majority of cases, the audio level needed on the PSP to match the perceived level of the internal tinnitus sensation will go down over a period of days and weeks. This is the principal result of CST and constitutes a “calming” of the tinnitus sensation.

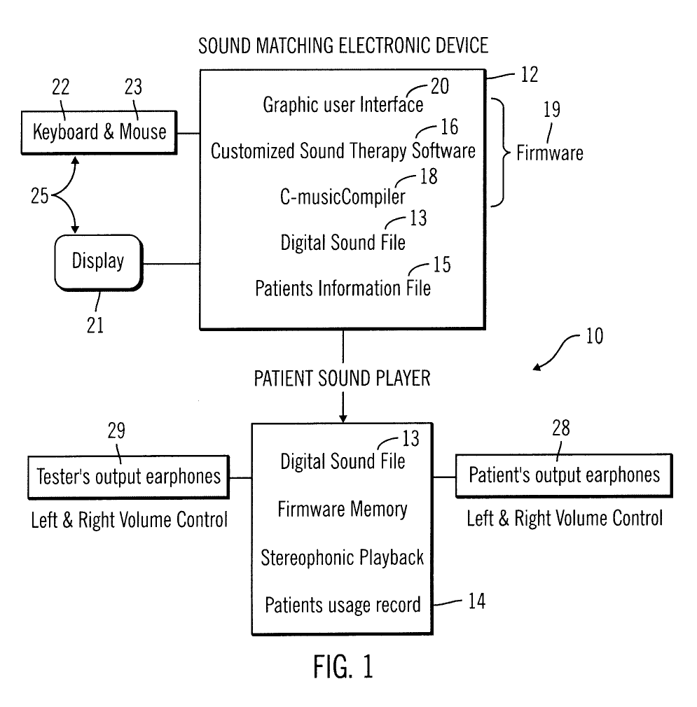

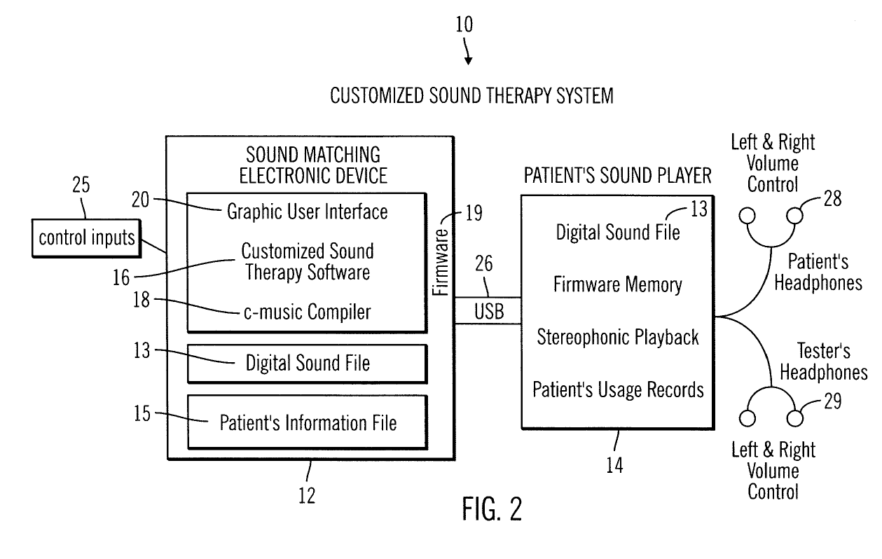

Referring to the embodiment illustrated in FIGS. 1 and 2, the CST System 10 includes a SMS 12, or an electronic device providing the function of a sound matching station, and a PSP 14.

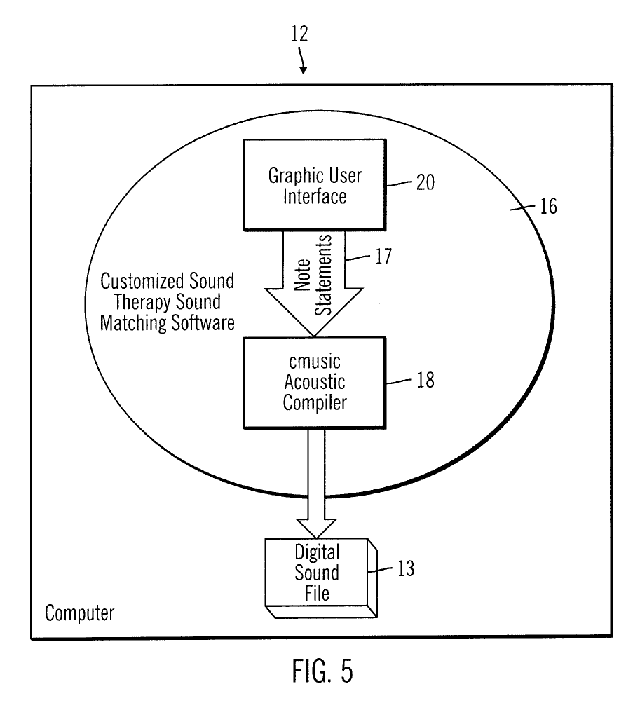

The intended user of the CST System 10 includes an audiologist or other trained professional or individual, who, using the GUI 20, identifies the patient's audio frequency by a unique matching process using forced choice procedures. The patient is the only one who can hear the tinnitus sound, and could judge the match. The software implemented CST application 16 in the SMS 12 uniquely identifies the tinnitus frequency the patient hears, and the cmusic compiler creates a replica sound of the patient tinnitus. A plurality of components of a person's tinnitus (e.g., up to twenty audio components) may be mixed together to produce a CST sound for use by the patient. The final CST sound is typically 3 minutes/180 seconds in duration (may vary from this depending on the intended therapy), and when played by the patient is automatically repeated. The person doing the matching, using the PSP 14, for example, may listen together with the patient to the matching sound the CST software produces, and the patient is comparing. When an acceptable match is made, the professional compiles the selected sound using the cmusic compiler and stores it in the patient's Digital Sound File, and copies it also to embed in the PSP 14. FIG. 5 is a schematic diagram illustrating the functional components of the SMS 12, generating a digital sound file 13, in accordance with one embodiment of the present invention. The sound matching or fitting session can be saved under a unique filename 15 (medical record numbers/identifiers consistent with HIPAA and any relevant patient privacy acts) for recall at a later date.

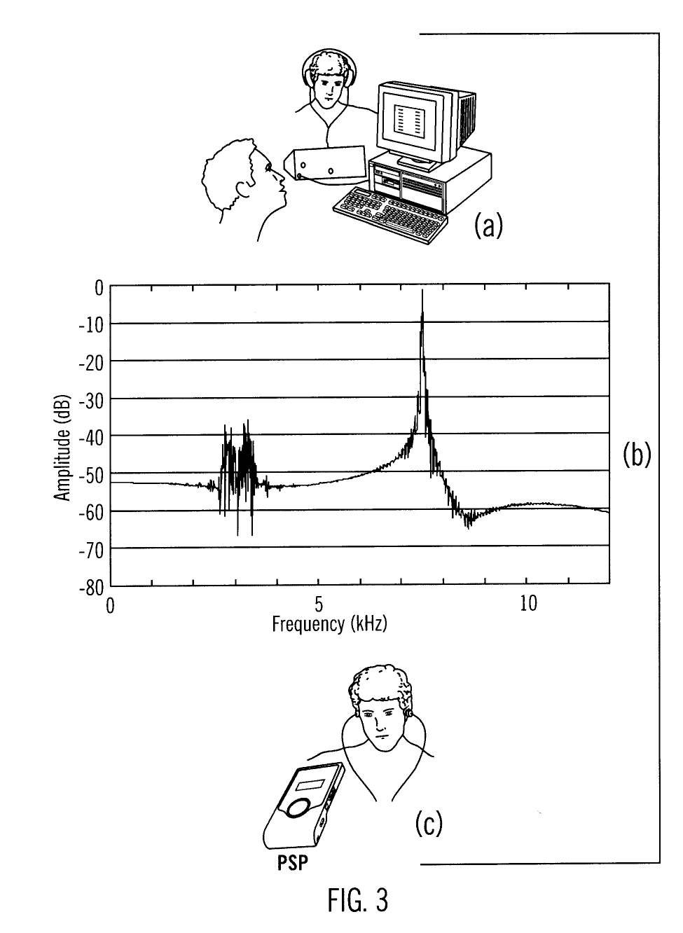

FIG. 3 is a pictorial representation of a CST session leading to generation of CST sounds. In FIG. 3(a), a tinnitus patient arrives at a clinic offering CST therapy. The patient is given ear/headphones connected to an interactive “sound matching station” or SMS that presents a variety of sounds and prompts the patient for feedback. Over one hour period, the audiologist uses the matching station to successfully replicate the precise tinnitus profile of the patient. (In FIG. 3(a), the SMS is illustrated as a desktop computer operatively connected to an audio player.)

Each person's tinnitus experience is unique and CST is programmed individually for each patient. FIG. 3(b) shows a spectrogram of a typical CST tinnitus habituation stimulus, showing two closely-spaced narrow band noises centered at 2800 Hz and 3225 Hz, and a very narrow band noise centered at 7417 Hz which is almost 40 dB stronger than the first two.

In FIG. 3(c), the unique sound file is then downloaded into a PSP 14 device that the patient wears a few hours a day over a period of several weeks. Over time, the vast majority of patients experience a notable reduction in the intensity of their tinnitus and in some cases a reversal of the disease.

CST—Sound Matching Station

The SMS 12 may be in the form of a digital processing device (e.g., a notebook, desktop computer, or other portable or non-portable digital processing device, which may be dedicated to sound matching functions and features, or include other functions and features such as those complementary to sound matching and tinnitus therapy). The SMS 12 includes a digital processor (e.g., a central processing unit (CPU), a mass storage device (e.g., hard drive), appropriate hardware and/or software operating system (e.g., Windows), and necessary drivers. Installed in the CST System 10 includes a CST application module 16 (e.g., implemented by software), a c-music acoustic compiler 18 (discussed below; the acoustic compiler is sometimes referred in the art as “pcmusic,” which is the version of cmusic for the Windows-based PC), and a GUI 20 designed to facilitate user interaction with the SMS 12 and related CST application 16. Essentially, the cmusic acoustic compiler 18 is an engine to synthesize the CST sounds for tinnitus therapy, and the GUI 20 is a tool that is used to design or develop those sounds. One or more of the CST application 16, cmusic compiler 18 and GUI 20 may be embedded in software, hardware and/or firmware 19, depending on the level of device integration for the SMS 12. The cmusic acoustic compiler 18 may be a part of the CST application 16, or may be a separate module interfacing and/or operatively coupled to the CST application 16. In addition to standard input/output (I/O) devices (or control devices 25) such as a display 21, keyboard 22 (or touch screen), a cursory pointing device 23 (e.g., a mouse), it has a high-quality sound output capable of playing digital audio files (e.g., .wav files). As external noise can interfere with the sound matching process, it is desirable for moving parts, if any, in the SMS 12 hardware (e.g., a cooling fan) to be as quiet as possible, and noise from such devices should preferably be isolated.

The Sound Matching Station (SMS 12) must be capable of generating candidate sounds for CST under control of the CST operator (physician, audiologist, or other qualified operator).

FIG. 5 is a schematic diagram illustrating the functional components of the SMS 12 (e.g., a computer) that generates a digital sound file 13 for the patient, in accordance with one embodiment of the present invention. According to one embodiment of the present invention, the following is an exemplary application code for the SMS 12:

File|New Session: initialize the whole session
File|Load Session: load existing session file (must end with “.ses”)
File|Save: save session information (including all components and
status of the current “Match Test” module) into a “.ses” file
File|Save As: save session as
File|Save Wave File: copy the already generated wave file (mix.wav
located in the program folder) into desired location.
File∥Session Info: notes for the current session. or patient
File|Exit: exit program
pcmusicScore|Edit Score: directly edit the current pcmusic score “mix.sc”
located in the program folder
pcmusicScore|Load Score: load score file (must end with “.sc”)
pcmusicScore|Save Score As: stored “mix.sc” to desired locations and
filenames
pcmusicScore|Run From Score: call pcmusic to run loaded or modified
score (“mix.sc” in the program folder)

The patient listens to these candidate sounds and responds to inquiries by the CST operator/tester about how to improve the approximation to the patient's internal tinnitus sensation. In that sense, the present CST matching procedure resembles eyeglass fitting (e.g., “Which sounds closer, A or B?” for CST, as compared to “Which looks better, this or this?” for eyeglass fitting).

In general, patients have tinnitus sensations that are matched by sounds having one or more “components.” The number of such components varies from patient to patient, but is typically one or two, though as many as a half-dozen or more have been encountered. Tinnitus is also rarely experienced equally in both ears—it is typically stronger on one side than the other. Every individual component may be sensed differently in terms of its left-right location. Components are typically “more” on one side than the other, but not necessarily exclusive to one side only. In general, therefore, each component is typically sensed as unequally strong on both sides.

So far, tinnitus components as experienced and described by patients have fallen into one of three categories: tonal, and two types of narrowband noise. The tonal category, which is quite common, is well-matched by a pure sinusoid of appropriate frequency. It should be noted that significant difficulties exist in matching the frequency of a sinusoid to the internal sensation in at least two ways.

The most significant difficulty is octave errors. Sinusoids whose frequencies are not too extreme have a well-defined musical pitch (such as Bb, F#, etc.), and patients often will judge a sinusoid with the same musical pitch class (i.e., note name) as being the same, even if it is one or more musical octaves lower (or higher). It is therefore important for the CST operator to check for octave errors by “bracketing” the sensation with sounds that are judged to be both lower and higher in pitch than the internal tinnitus sensation, if possible.

The second significant difficulty in matching sinusoids to tonal tinnitus sensations is that “normal” frequency-matching cues, such as beats, are not present when the sinusoid frequency gets close to the internal sensation. Patients typically evince a slight confusion when the sinusoid approaches very closely to the internal sensation, often describing a kind of “occlusion” of the sensation by the sound, or vice versa. Sometimes the internal tinnitus sensation disappears altogether, at least momentarily, when the candidate sound matches it or approximates it very closely.

An important requirement for the SMS 12 during a sinusoid-type component match is the abilities to produce a sequence of one or more sinusoids with precise, specifiable frequencies and amplitudes. These are adjusted by the CST operator until the patient is unable to detect a difference in the frequency of the sinusoidal component and a component of the internal tinnitus sensation. If a tinnitus sensation consists of multiple sinusoidal components, the usual practice is to proceed from the most prominent to the less prominent ones. Each component is considered matched when the patient cannot suggest further improvements in the frequency of a given sinusoid. For most patients, this involves making adjustments to the frequency of the synthesized sounds as small as one or two just-noticeable differences (jnd's) around the relevant frequencies.

Tinnitus sensations often match narrowband noise sounds rather than sinusoids. It is also not uncommon for a patient to experience a tinnitus sensation that matches both sinusoidal and a narrowband noise sounds mixed together. In terms of the SMS 12, such a sensation would be matched by a sinusoidal component mixed with a second, narrowband noise component.

Two types of narrowband noise components have been commonly encountered. “Type I” noise is generated using a special random algorithm developed in the context of computer music sound synthesis (see FIG. 11). “Type II” noise is simple white noise, filtered by a second-order, normalized digital filter, described below.

The noise source for Type I noise is a digital signal consisting of a waveform with a random value chosen every tau (τ) seconds, with linear interpolation used to fill in the samples between random values. This results in a signal with a spectrum having a 0 Hz centroid, and sides lobe that drop to zero amplitude at harmonics of the frequency 1/π Hz. The first side lobe has a peak amplitude approximately 24 dB below the centroid. Successive side lobes fall off in amplitude at the rate of about 12 dB per octave. The value of τ therefore controls the bandwidth of the noise signal.

This noise spectrum is easily shifted to an arbitrary center frequency, fc Hz, by convolving its spectrum with a sinusoidal spectrum at fc Hz, easily accomplished through a waveform multiplication (four-quadrant modulation). This technique provides both bandwidth and center frequency controls for Type I noise, both of which must be adjusted to match the patient's internal tinnitus sensation.

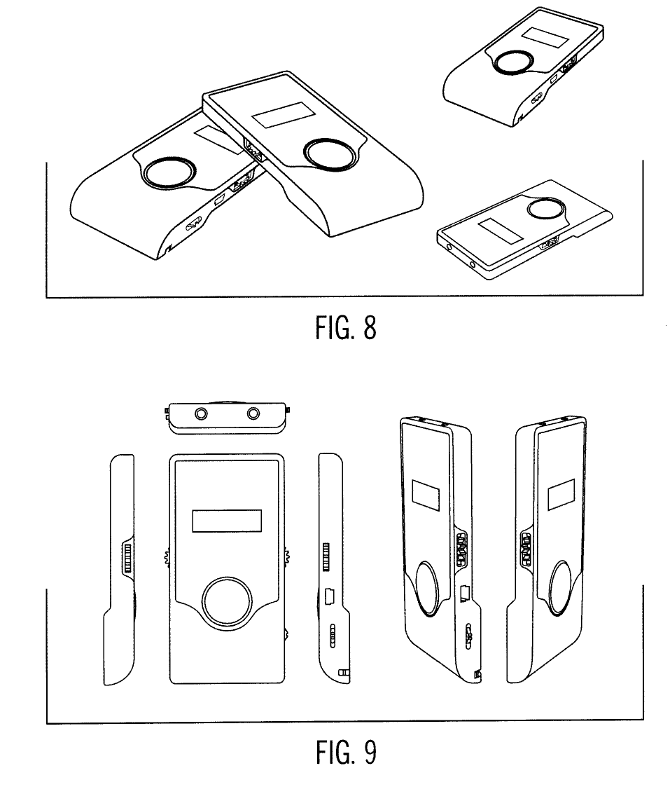

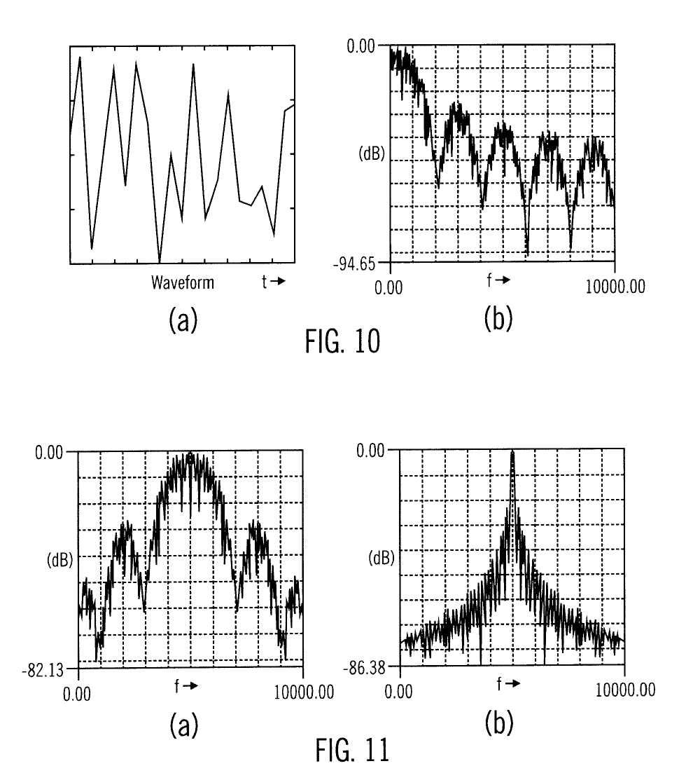

Type I noise (see FIGS. 10 and 11) has a “rougher” quality than simple band pass-filtered white noise. Its utility is that it is often judged by patients to be close in quality to noise components of their internal tinnitus sensation. FIG. 10(a) illustrates a waveform consisting of random values chosen in the range ±1 every 0.5 ms (i.e., at a rate of 2000 Hz), with linear interpolation between adjacent points. FIG. 10(b) illustrates the associated (normalized) right-half amplitude spectrum, showing a centroid at 0 Hz, and successive side lobes. FIG. 11(a) illustrates spectrum from FIG. 10 centered at 5000 Hz by spectral convolution with a 5000 Hz sinusoid. FIG. 11(b) illustrates similar to FIG. 10, with 1/π=200 Hz, centered at 5000 Hz.

Type II noise is bandpass filtered white noise. It has a “smoother” quality than Type I noise. The bandwidth may be adjusted sufficiently narrow to provide a continuum between tone-like and noise-like sounds. It is produced by connecting a white noise source (typically, a linear congruential source of pseudorandom numbers) to a second order filter with normalized gain. Gain normalization is necessary because simple two-pole filters exhibit a significant increase in peak gain at the center frequency as the bandwidth is narrowed (corresponding to moving a pole-pair close to the unit circle on the complex plane. FIG. 12 illustrates frequency-dependent gain of a two-pole filter for pole radii of 0.0, 0.7, 0.8, and 0.9. The center frequency of this filter is set to one-half of the Nyquist rate.

The simple two-pole filter shown in FIG. 12 has the filter equation:

y(n)=a₀−b₁y(n−1)−b₂y(n−2)

which has the transfer function:

$$H ( z ) = \frac{{a}_{0}}{1 - 2 R cos ϕ {z}^{- 1} + {R}^{2} {z}^{- 2}}$$

where the center frequency of the filter is determined by pole angle φ and the bandwidth is determined by pole radius R (≦1). In order to normalize the peak gain of this filter to unity at most frequencies, it is possible to introduce two antiresonances (zeors) at z=−1 and z=1, corresponding to 0 Hz and the Nyquist rate, respectively [Smith and Angell, 1982]. Scaling the resulting transfer function by a factor of (1−R) normalizes the peak gain of the filter to unity, as follows.

$$H ( z ) = ( 1 - R ) \frac{1 - {Rz}^{- 2}}{1 - 2 R cos ϕ {z}^{- 1} + {R}^{2} {z}^{- 2}}$$

This corresponds to the filter equation:

y(n)=G[x(n)−Rx(n−2)]+b₁y(n−1)−b₂y(n−2)

where
R˜e^{-πB/S}
G=1−R
b₁=2 R cos(2πf_{c}/S)
b₂=−R²
   Here, S is the sampling rate, fc=Sφ/(2π) Hz is the center frequency, and B˜−S ln (R/π) Hz is bandwidth. This second-order filter allows the center frequency and bandwidth to be varied without regard to filter gain, though the overall amplitude of the filtered noise tends to decrease sharply for very narrow bandwidths.

To summarize, the SMS 12 must be capable of producing an arbitrary number of components, each of which is tonal, or Type 1 or Type II noise. Each component should be balanced arbitrarily between left and right stereo channels. Finally, the resulting sound should be monitored directly through the PSP 14 by both the patient and CST operator to insure that the therapeutic sound used by the patient matches the sound obtained during the CST fitting operation.

CST—Portable Sound Player

The PSP 14 has several requirements (such as volume limitation, playback session logging) that are not found in a typical audio player. Unlike a general audio player, the PSP 14 does not need to produce sound at high volume levels, such as those suitable for general music listening. In fact, because the patient will be listening to a customized sound for many hours at a time, it is highly desirable that the PSP 14 be limited to producing sounds at fairly low listening levels. In order for effective habituation to occur, the customized sound should be audible, but not loud enough to mask the tinnitus sound itself. Therefore, the volume settings that tinnitus subjects use on their PSP 14 give an estimate of the intensity of the tinnitus. The audio power involved is highly frequency-dependent, but in any case should not exceed the patient's threshold (e.g., approximately 80 dB SL (sensation level, i.e., dB above patient's threshold)). It has been found that most tinnitus sensations are well-matched by sounds in the frequency range from about 3 kHz to 10 kHz. However, tinnitus sensations matching sounds as low as 50 Hz and higher than 14 kHz have been observed. Therefore, the PSP 14 needs to be capable of generating frequencies covering the entire audible range (about 20-20,000 Hz) at low distortion.

Referring to FIG. 2, the detachable/portable PSP 14 is operatively coupled to the SMS 12 station via a suitable interface 26 (e.g., a USB interface), is connected, wired or wirelessly, to audio output transducers 28 and 29 (e.g., headphones) for both the patient and the CST operator during the matching session, then detached for patient's use during habituation therapy. The SMS 12 could be configured to support multiple PSPs.

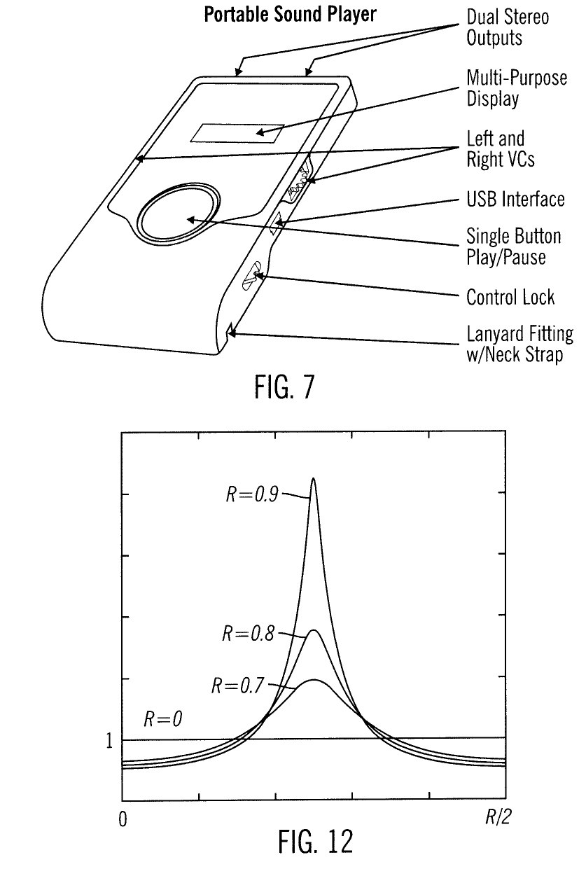

In the embodiment illustrated in FIGS. 7, 8 and 9, the PSP 14 includes the following structures, features and functions:
A convenient on-off control to preserve battery life, and a long battery life between recharges (at least 8 hours of playback between charges).

The PSP for CST is required to operate for longer than a typical MP3 player in continuous stretches, perhaps, 2 or 4 or 6 or 8 hours at a time, though it is desirable to minimize this time for purposes of patient convenience. Conservation of battery power is desirable, and could be essential to obtain optimal treatment.
A convenient play-pause button.

Patients being treated for tinnitus are often elderly and less “tech savvy” than typical younger music player (e.g., Apple ipod) users. A simple user interface is not simply desirable, but critical to patient effectiveness.
A control hold (lock-out) button to prevent accidental change of settings.

This is important for CST patients, as treatment settings must be preserve and continuity of the settings maintained throughout the rather long period (e.g., 2 to 8 hours) for a session of CST.
Continual, repeated playback of a single recorded sound of a preset duration (e.g., at least 5 minutes duration).

Patients typically play back repeated sounds of several minutes for several hours per day. Three minutes seems enough to allow patients to remain unaware of repetitions. The treatment sound fades in and out every three minutes. Typical repetitive CST sounds may be 3 minutes in duration, but future development of longer CST sounds may be 5 minutes in duration. Five minutes for the player would be a conservative safety measure to prevent awareness of repetition.
Sterophonic playback to allow for differential playback in each ear.

Therapeutic CST sounds can have identical signals applied to each ear. But patients often have differing hearing capabilities in each ear. Stereo playback allows for differing sounds to be played in each ear as needed on an individual basis. Stereo playback also has other well-known characteristics adding to apparent sound realism. This has potential use for all sound playback. Stereo reproduction allows for incorporation of arbitrary binaural sounds that may address other aspects of tinnitus, such as depression or anxiety.
A balance control to allow the relative loudness in each ear to be adjusted.

Balance controls are lacking in modern portable players, such as the Apple iPod. Balance controls apparently are missing in most commercial players because most consumers don't care. They are important for CST purposes. Many, or most, tinnitus patients have different abilities to hear in each ear. The ability to control the loudness in each ear is therefore a wise safety precaution.
A convenient volume control and a multi-purpose display giving a numerical readout of volume level.

Volume controls with precise readouts of settings are desirable to CST treatment, as it is these precise settings that measure treatment efficacy. Instead of a balance control, separate volume controls for L and R channels would provide the separate volume level control for both ears. The volume controls should be able to control relatively fine increments (e.g., 0.5 dB), compared to increments of 3 dB for a typical MP3 digital player. And the range of the volume controls should be able to deliver at least 5-10 dB above hearing threshold for tinnitus. A display with large fonts for displaying information of various operating parameters, such as numerical readout of volume settings, would be desirable if the PSP is intended for use by an elderly patient.
Playback volume limitation limited to a certain volume level.

CST involves long-term exposure to certain sounds. The maximum volume level of the PSP is therefore limited to the precise levels that are specified by OSHA (http://www.osha.gov/pls/oshaweb/owasdisp.show_document?p_table=STANDARDS&p_id=973 5) for long term (8 hour) sound exposure. This prevents the PSP from possibly being hazardous when used as directed.
An internal date and time of day clock to allow internal logging of playback times and volumes; and an internal monitoring software to log playback dates, times, volumes, etc.

A principal means of determining the efficacy of CST treatment is the gradual trend of playback volume to match the tinnitus sensation over time. The PSP is specifically designed to track this aspect of patient usage. The duration of the use for each treatment depends on the tinnitus symptoms. For some patients, relatively minimal usage for patients has provided significant results, such as two or so hours per day for three or so weeks. We also recommend rematching of sound when the patient begins to perceive a difference between the treatment sound and their perception of their own tinnitus sensation. The patient's changing of their personal sound loudness data to match their subjective sensation can be principle evidence of the effectiveness of CST. Each and every time the patient rehears the repeated (e.g., 3-minute) sound, or each and every time she adjusts it, the date and time of day are recorded.
Provision for connection to multiple transducers in order to allow monitoring by SMS operator during sound customization or at other times.

Independently-buffered audio outputs from the PSP allow two things: (1) simultaneous monitoring of the sound by both the patient and the CST provider based on identical audio output characteristics of the audio player in the PSP, and (2) no change in the sound when one or the other of the patient or provider has earphones plugged into the same PSP.
USB or other convenient interface to the SMS allowing exchange of sound and logging data, which should include playback times and volumes, and other patient data, such as ID and arbitrary text notes, such as sound specifications.

This allows for tracking the progress of patients undergoing the CST treatment. One additional aspect of the PSP is that it can be used as a USB-driven sound output device, in order to facilitate the CST sound match process. The PSP can be used as an audio interface connected to the SMS, with built-in audio playback electronics in the PSP tuned for CST, so that the patient and CST operator can both hear from the same playback source audio files received from the SMS. The SMS either plays the audio information directly (to both the patient and CST provider) through the PSP using the USB interface and dual audio outputs, or it uses the USB interface to copy the audio data to the flash memory of the PSP, from it which can also be played independently (and dually) by the PSP. The importance of this is that the PSP playback sound in all cases is absolutely identical, since the same audio playback hardware in the PSP is being used as the reference player.
Transducers for one or both ears

It would be desirable to connect the transducers wirelessly to the PSP. Wireless connection at high audio quality is required for CST for tinnitus treatment.
High Quality Audio requirements

The sound playback should be at high quality, e.g., at a minimum essentially that of standard red book CD audio, i.e., 16-bit linear PCM stereo sampled at 44,100 samples per second per channel. The analog audio output circuitry needs to be of high quality, with noise and distortion characteristics on the order of those of high quality digital music player, such as MP3 players (e.g., the Apple iPod player) or better. The analog audio output needs to be able to drive at least two sets of transducers with independent volume settings (e.g., left and right VCs shown in FIG. 7), allowing simultaneous sound monitoring by both the patient and the CST operator based on identical audio characteristics of the two outputs of the PSP.
Memory requirements

Assuming the sound is recorded as standard 16-bit linear PCM stereo audio (1.411 Mbs), the audio storage requirements are on the order of 64 MB. Additional storage for software and data logging may double or quadruple this. Firmware memory requirements are hardware-dependent, and preferably updatable to allow for future improvements.
A lanyard fitting with neck strap, to provide convenience to facilitate patient “wearing” the PSP device with ease, with less interference to the patient's daily routine.

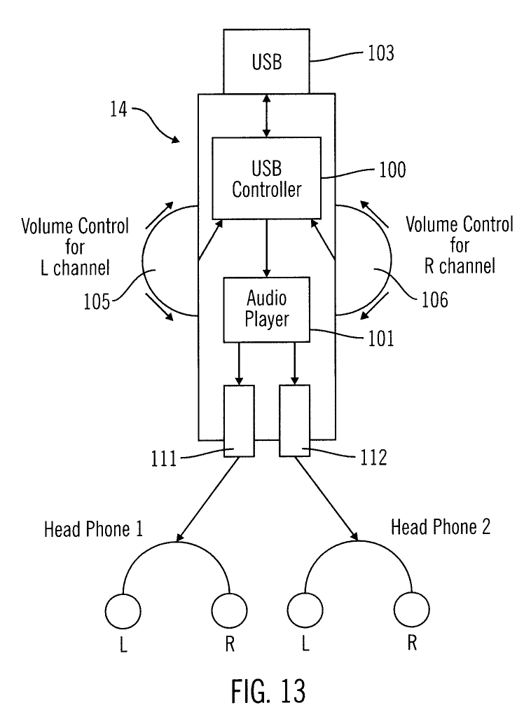

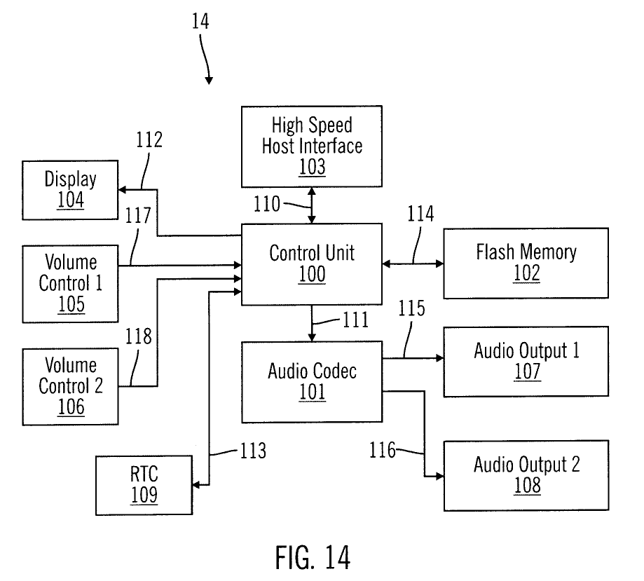

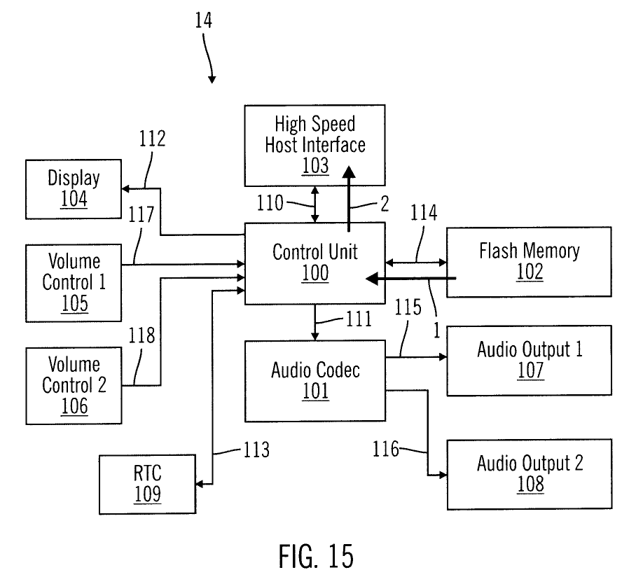

FIG. 13 is a schematic diagram of the components of the PSP system 14. FIG. 14 is a schematic block diagram of the components of the PSP system 14. The PSP system 14 includes an audio player which includes an audio processing unit (which may include an audio codec 101) communicating with a high speed host interface 103, such as a USB interface via a control unit 100, which may include a USB controller. The USB interface 103 is used to make external connections to the SMS, for example. The control unit 100 also controls the repeat playback of a single recorded sound for a predetermined duration. Separate volume controls 105 and 106 control the volume level for L and R channels, which outputs from the audio codec 101 via audio outputs 107 and 108. FIG. 13 shows two independently buffered and identical outputs 111 and 112 to two sets of transducers (e.g., stereo head phone 1 and head phone 1). Each buffered output 111 and 112 includes a pair of audio outputs 107 and 108. (FIG. 14 shows only one of the two outputs 111 and 112, comprising a pair of audio outputs 107 and 108). A flash memory 102 is provided, which is accessible by the control unit 100. The control unit 100 also controls an RTC 109.

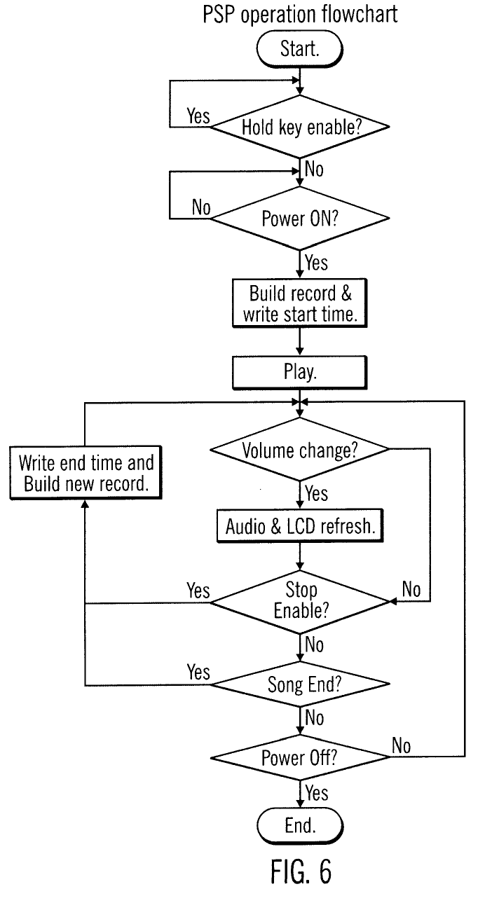

FIG. 6 is a schematic flow diagram illustrating the operation of a PSP used in the CST System 10, in accordance with one embodiment of the present invention.

When the patient uses the PSP 14, it records automatically the patient's usage, i.e., it records (by building a record) the date and time when the PSP 14 was turned on and off, and the sound volume used at that time. This information can be downloaded at the next visit onto the SMS 12 and reviewed.

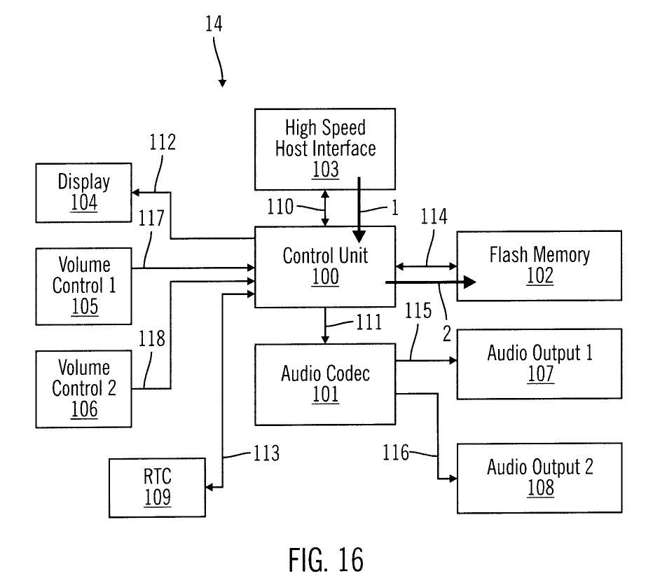

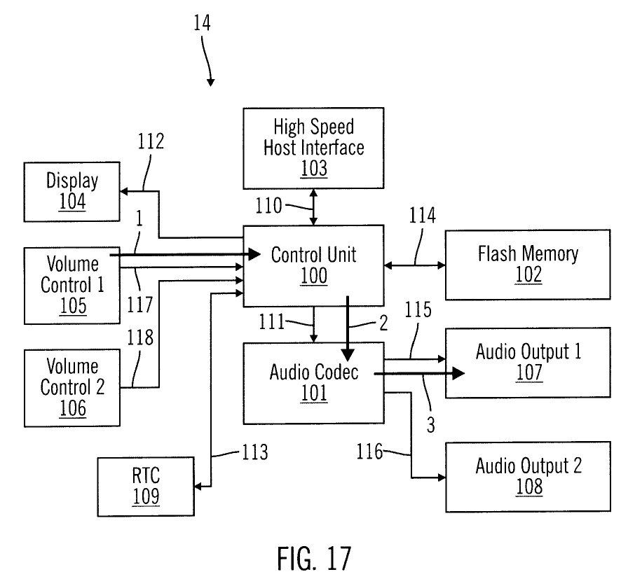

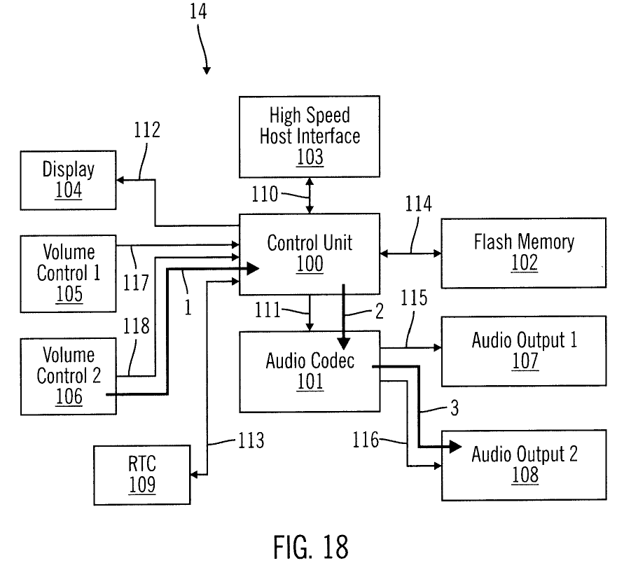

FIGS. 15-19 are schematic diagrams illustrating various operations of the PSP system 14. FIG. 15 is a schematic block diagram illustrating the operation of uploading text file from flash memory to the external SMS (e.g., a PC). The data in flash memory 102 is routed by the control unit 100 towards the high speed host interface 103. Wave/music from the SMS/PC is downloaded from the high speed host interface 103 by the control unit 100 to the flash memory 102, as illustrated in FIG. 16. In FIG. 17, the volume level change operation of one of the channels is by adjusting the volume control 105, getting the change status from volume control 105 to the control unit 100, and providing control of the audio wave output level through the audio codec 101. The audio output 107 and/or audio output 108 receives the output from the audio codec 101. In FIG. 18, the volume level change operation of the other channel is by adjusting the volume control 106, getting the change status from volume control 106 to the control unit 100, and providing control of the audio wave output level through the audio codec 101. The audio output 108 and/or audio output 108 receives the output from the audio codec 101.

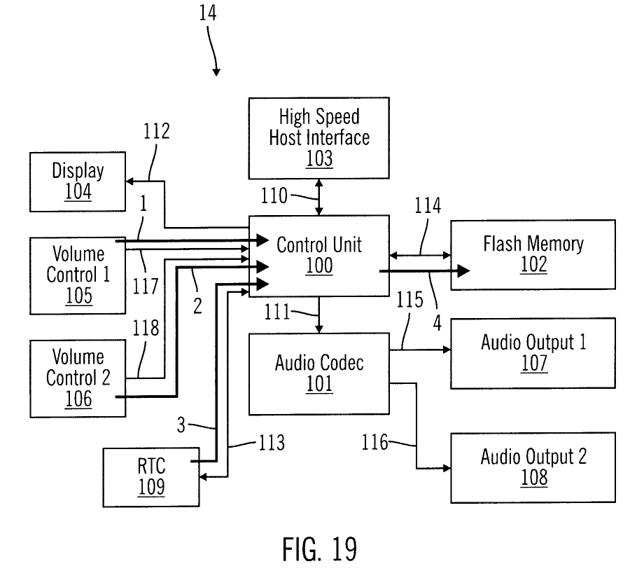

In FIG. 19, the logging operation is schematically illustrated. Date and time are retrieved by the control unit 100 from RTC 109. Volume control levels of both volume controls 105 and 106 are retrieved by the control unit 100. The retrieved date/time and volume control levels are stored in the flash memory 102.

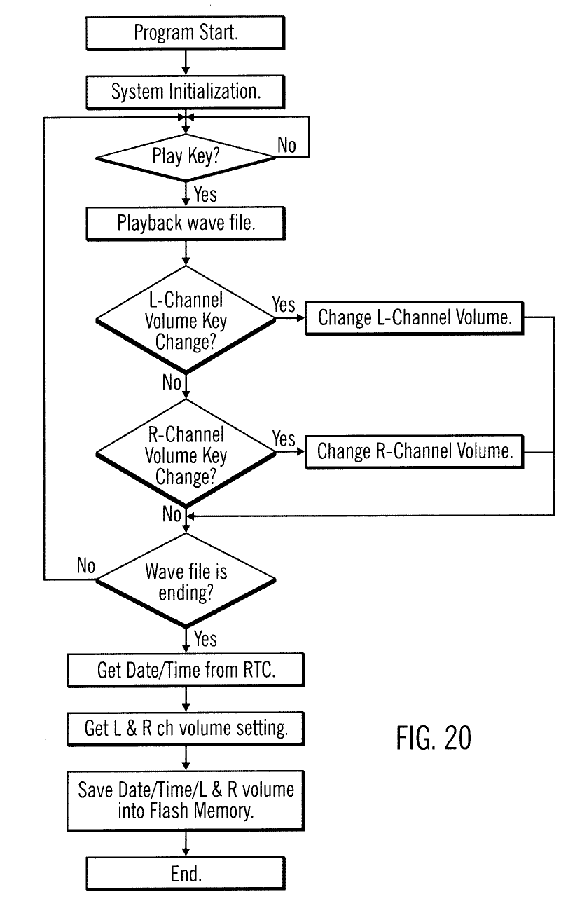

FIG. 20 is a schematic flow diagram illustrating the operations of the firmware in the PSP system 14, in accordance with one embodiment of the present invention.

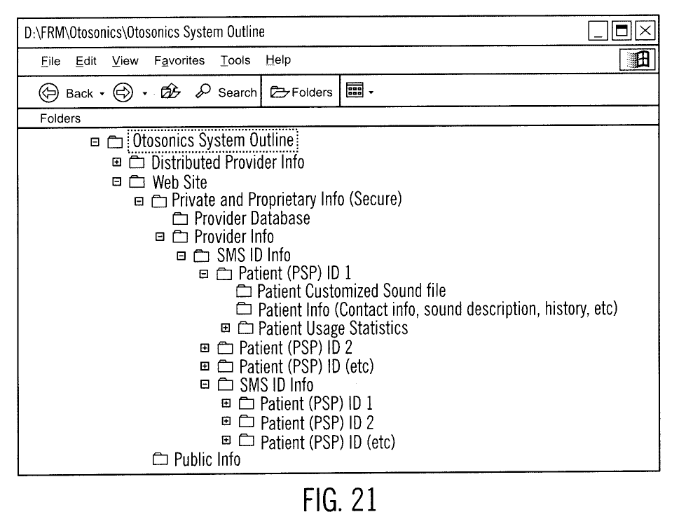

FIG. 21 is a screen shot of the display at the SMS, which illustrates a folder structure of information uploaded to and downloaded from the SMS, in accordance with one embodiment of the present invention.

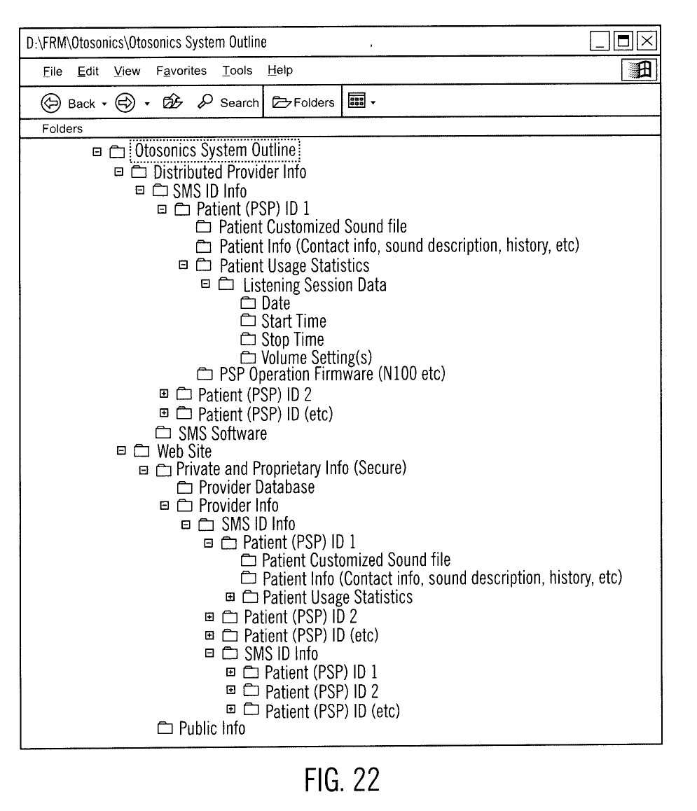

FIG. 22 is a screen shot of the display at the SMS, which further illustrates a folder structure of distributed provide information, including patient usage statistics.

Acoustic Compiler

The audio compile referred herein as “cmusic” is designed and implemented by F. Richard Moore, and is fully documented in his book Elements of Computer Music (Prentice-Hall, 1990). The “cmusic” acoustic compiler 18 is a software implemented application having the features and functions of an acoustic compiler adapted for use in the inventive CST System 10. Acoustic compilers have been used in the field of computer music synthesis for several decades. By definition, an acoustic compiler turns a description written in the “source” language it defines into a corresponding digital audio signal that can be stored in a suitable computer file and then played back as an audible sound using standard digital audio playback methods (typically, sound cards on standard computers, or more specialized playback systems, such as portable sound players). The cmusic acoustic compiler 18 defines a source language that is as flexible and general as possible with respect to its domain. It provides various “building blocks” out of which virtually any sound can be specified, and therefore synthesized.

More specifically, the acoustic compiler takes as input digital file containing a textual description of one or more sounds to be synthesized. This textual description is written in an input language defined by the particular acoustic compiler in use (in this case it is the cmusic input language). Statements in this language describe the detailed characteristics of the sound signal to be synthesized, such as its component content, where each constituent component may have such parameters as frequency, relative amplitude, phase, waveshape, and so on. The acoustic compiler then “realizes” the input by generating the corresponding sound signal, storing the result on a digital audio data file. The digital audio data file is essentially a digital “recording” of the specified sound. This digital audio file is then converted into sound using a digital-to-analog conversion system, which may be incorporated into the computer itself, or located elsewhere, such as in an external digital audio player.

Various compilers are typically differentiated in terms of which types of sounds they make convenient for specification (just as Fortran and C compilers make different kinds of algorithmic processes more or less convenient to specify). The cmusic acoustic compiler may be deployed as an application in the SMS 12 or other types of free-standing computers. In the illustrated embodiment, the particular version used for CST uses the PC version of the cmusic acoustic compiler, which is implemented as a console application (command-line) program run via a command window under Microsoft Windows.

The acoustic compiler, cmusic application, is used by a CST provider (typically an audiologist) to synthesize sounds that are candidates for matching a patient's tinnitus sensation. Each time the specification of the sound is changed by the provider; a new textual input file is created. The cmusic application is then run to turn this textual description into the corresponding sound signal. After a multiple-trial, forced-choice procedure (similar to choosing eyeglass lenses), the best match is identified. The cmusic application is then used to synthesize a signal of longer duration (typically about 3 minutes) which can be downloaded into a portable sound player carried by the patient. The patient then can listen repeatedly to this sound using the auto-repeat feature of an audio player in order to receive the CST therapy.

CST Graphical User Interface

The cmusic compiler was originally specified to make the specification of sine tones and two types of noises that have been found to be beneficial in the treatment of tinnitus. The cmusic source language is quite complex, however, and not well-suited to use by non-specialists. In the illustrated embodiment, the GUI 20 specifically designed around the needs of CST provides a simplified way of running cmusic for persons not familiar with acoustic compilers, such as the typical audiologist. One or more versions of a GUI have therefore been devised to make the specification of these sounds, in forms suitable for tinnitus therapy, available to users who are expert in tinnitus therapy, but not expert in the use of acoustic compilers. This GUI 20 simply runs the cmusic application on behalf of the user, but in no way affects the underlying sound synthesis procedure. In CST, the user manipulates the GUI 20 to converge on sounds matched to the tinnitus sensation of a patient. The GUI 20 produces statements 17 (see, FIG. 5) in the cmusic source language, which are fed internally to the cmusic program, which in turn produces the specified digital audio signal corresponding to the desired sound. Once it is determined that this sound properly “fits” the tinnitus sensation of the patient, it is downloaded into a portable player for use by the patient.

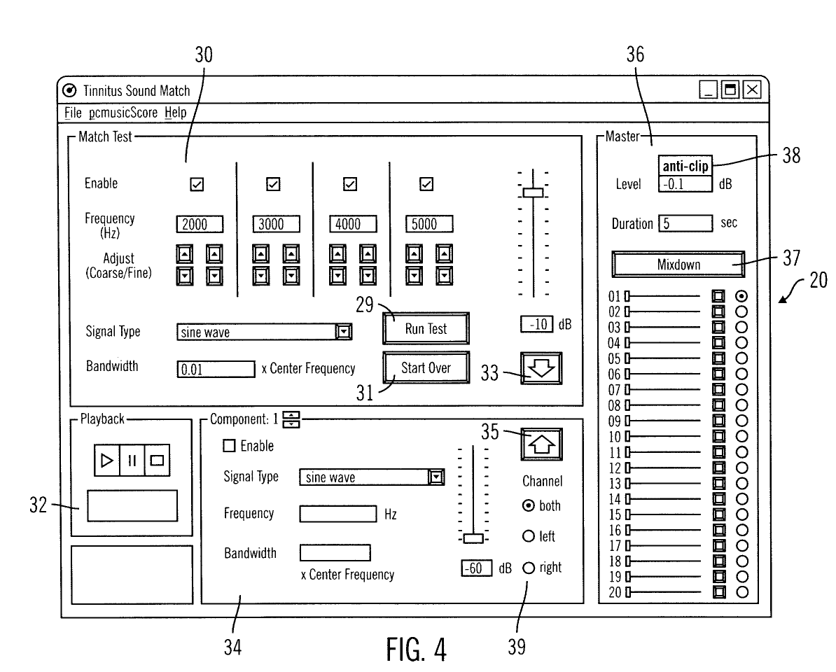

Referring to FIG. 4, the GUI 40 is divided into four main sections:

1. “Match Test”

This GUI section 30 is used for matching sounds to an individual component of a patient's tinnitus (depending on the patient, there may be only one component). Signal type, frequency, bandwidth (if appropriate), and level (amplitude) of the component can be controlled. In the illustrated embodiment, the GUI allows a maximum of four different test sounds to be compared in fairly rapid sequence.

Buttons:
a. “Run Test” 29 generates the synthesized sound “test.wav” file and plays it back as indicated in the “Playback” section.
b. “Start Over” 31 will bring the entire GUI back to its initial settings.

After each test, the result can be transferred into a selected component of the eventual mixdown of sounds with a down arrow 33. If more than one of the frequencies is enabled, the right-most among them will be transferred into the selected component

2. “Playback”

This GUI section 32 is used to control playback of the most recently synthesized sound.

3. “Component”

This GUI section 34 displays one component of the mixdown sound at a time. The specific component displayed is indicated with a radio button in the “Master” section 36. Properties of each component include: signal type, frequency, bandwidth, level, and channel assignment (left, both, or right).

The button 35 with an up arrow enables the user to transfer the selected component back to the “Match Test” section for further test and adjustment.

4. “Master”

This GUI section 36 provides a mixer of all the components of a person's tinnitus. It allows a plurality of components to be mixed (e.g., up to a maximum of twenty components). The duration (in seconds) of the component mixture can be specified.

The “Mixdown” button 37 synthesizes a mixture of all components and generates a digital sound file (e.g., a “mix.wav” file) located at the program folder or a user specified folder.

The mixer provides level on/off (i.e., mute or not) control for each of the component tracks (e.g. up to the maximum twenty component tracks). The option box in the right-most column of the mixer decides which component to be displayed in “Component” GUI section as the “current component.”

“Anti-clip” 38 permits user specifying the duration for the threshold level to avoid clipping.

### ILLUSTRATIVE EMBODIMENTS

The matching process starts with taking a routine audiological patient history of the prescreened patient targeted to determine the general nature of their tinnitus sensation. Obtaining this history has several purposes: to establish an initial starting point for the matching process, to introduce the patient to the matching process and to act as further screen for indications of active ear disease (preliminary medical evaluation must always be performed prior to initiating CST and results of this evaluation should be available to the audiologist/professional performing the CST procedures). Indications of active ear disease may include, but are not limited to: tinnitus that varies widely in frequency or intensity, pulsatile tinnitus or unusual tinnitus pitches. Throughout this history, additional information that may aid in the matching process may also be discovered (e.g., patient's level of sophistication regarding description of sounds).

Case Study 1: Single Tone Tinnitus

In the simplest case, a patient will have a tinnitus sensation that is precisely matched by a pure tone/sine wave at a specific frequency. Matching the tinnitus sensation then requires only the discovery of the frequency of the pure tone/sine wave that is closest to the patient's tinnitus frequency. Using the GUI 20 shown in FIG. 4, the CST application 16 in the SMS 12 allows up to four test tones to be generated. The CST user/provider can set these tones to any frequencies (both coarse and fine frequency adjustment controls are provided and arbitrary frequencies may simply be typed into the frequency sub windows). The CST matching procedure is based on two sample “forced choice,” wherein the Provider plays two tones for the patient and asks which of them is “closest” in pitch to the patient's tinnitus sensation. The choice of test tones used is based on the provider's judgment according to prior information from any tinnitus evaluations performed and the description of the tinnitus by the patient (“very high pitch,” “like a cricket,” “like the sound that TV sets make,” etc). It is helpful if the patient can describe a test tone as “higher” or “lower” in pitch than a given test tone, or even better if the tinnitus sensation lies “in between” test tones. The “in between” assessment by the patient is especially useful in avoiding octave errors in pitch. Whenever such basic pitch determination is undertaken, it is especially important to “bracket” the tinnitus sensation in between two test tones to avoid octave errors.

Once the pitch of a tinnitus sensation has been bracketed using test tones, a useful technique is to gradually decrease the ambitus (pitch difference) between two tones until an exact (or unimproveable) match is achieved. Depending on the frequency range and the patient's ability to compare the pitches of test tones with the tinnitus sensation, this process can be repeated until an exact match is achieved, or the patient is unable to decide between two test tones. The latter can occur when two tones lie within an approximate just-noticeable difference (jnd) in frequency around a given frequency (typically less than about 0.5% frequency change).

Once a good candidate test sound is found, it can be transferred to the component window for further adjustment. Up to twenty components may be mixed together to produce a CST sound for use by the patient. The final CST sound should typically be set to 180 seconds in duration before saving. The fitting session should be saved under a unique filename (e.g., consistent with the Patient Privacy Act) for later recall. All files are saved in the CST program's installation directory. All sound files are stored under the name “test.wav” or “mix.wav” unless it is saved under another name (typically the same as the session name with a “.wav” extension).

In the Component sub window 34, the CST provider can elect to cause the tone to be played equally in both channels, or only in the left or right channel, by selecting one of the options 39. If it is desired to play the tone in both channels, but louder on the left or right, the same sound can be used for two Components, one on the left and one on the right. The loudness level of both components can be adjusted separately. Such adjustments can be made using Master Mixdown durations of 5 or 10 seconds until the correct left-right balance is achieved. The therapeutic tone can then be fabricated by setting the duration to 180 seconds (typical) and synthesizing the tone. Depending on the number of components in the final mixdown, the level of individual components may need to be reduced to prevent amplitude clipping during the mixdown. If only one or two components are used, the individual components amplitudes can be set to, e.g., −6 dB or less (they will typically be much less than this in any case). If more components are used, the maximum allowable component level should be reduced by a predetermined level, e.g., 6 dB, every time the number of components doubles.

All the settings displayed on the GUI 20 can be saved for later recall under a session name (session names should be consistent with HIPAA and any relevant patient privacy acts). This allows the sound match procedure to be interrupted and continued at a later time, or for a previous setting to be recalled for further adjustment that may be stored under a new version name (such as Pt1ver2 or “Patient 1, version 2”).

In any case, the final mixdown sound is stored by saving it as a .wav file with a name corresponding to the session under the File menu. Once the .wav file is created, it can be transferred to the PSP 14 for use by the patient.

The therapeutic sound file is copied onto the PSP 14, which the patient can then listen to repeatedly. Since it is expected that the volume level required to just barely match the tinnitus sensation will decrease over time, the starting level to achieve this match should be between one half and full scale on the PSP 14 (component amplitudes for the mixdown can be adjusted to insure this).

The CST provider should provide detailed instructions to the patient, as well as obtaining any necessary release or other forms. At the end of the sound-matching session, the patient should have a PSP 14 containing a sound that matches his or her tinnitus sensation as closely as possible.

Case Study 2: Noise-Band and Multiple Component Tinnitus Sensations

Not all tinnitus patients experience single-tone sensations. Some patients experience a tinnitus sensation that is more adequately matched by a narrowband or a broadband noise, and some patients have a combination of one or more pure tone sensations combined with one or more noiseband components. The CST program provides two types of noiseband components, noise Type I (band limited), and noise Type II (filtered white) noise. In essence, noise Type I sounds a little “rougher” while Type II sounds a little “smoother.” Patients sometimes describe noise Type I as more “scraping” or “cricket-like”, while noise Type II is more like a “hiss” or “rushing stream.” Both of these noises can be generated by the CST application in the SMS 12. In addition to a basic, or center, frequency, each of these noises has a “bandwidth” adjustment that changes its sound quality. A very small (or “narrow”) bandwidth makes these noises more tone-like (i.e., more like the pure tone/sine wave tones discussed previously), while a larger (or “broader”) bandwidth makes these noises more similar to a “rushing steam”, “wind” or “running water” sound.

The procedure for matching a noiseband to a patient's tinnitus sensation is essentially the same as described previously for pure tones/sine waves, except that there is an additional parameter of bandwidth to adjust. Noise Types I or II can be transferred into the Component sub window in the same manner as for pure tones/sine waves. They may be assigned component numbers if there is more than one present in the patient's tinnitus or if the left and right ears have differing tinnitus sensations.

It is not uncommon to find that a patient's tinnitus sensation consists of a combination or one or more pure tone/sine wave tones and one or more noiseband sounds of noise Type I or II. The CST application provides for up to 20 components to be mixed together to produce a sound that best matches a patient's tinnitus sensation. As mentioned previously, when more than a few components must be mixed together, it may become necessary to lower the amplitude of all component sounds in order to avoid sound distortion due to “clipping.”

The process and system of the present invention has been described above in terms of functional modules. It is understood that unless otherwise stated to the contrary herein, one or more functions may be integrated in a single physical device or a software module in a software product, or a function may be implemented in separate physical devices or software modules, without departing from the scope and spirit of the present invention. It will be further appreciated that the line between hardware, firmware and software is not always sharp.

It is appreciated that detailed discussion of the actual implementation of each step that comprises the process is not necessary for an enabling understanding of the invention. The actual implementation is well within the routine skill of a programmer and computer engineer, given the disclosure herein of the system attributes, functionality and inter-relationship of the various software and hardware components in the system. A person skilled in the art, applying ordinary skill can practice the present invention without undue experimentation.

While the invention has been described with respect to the described embodiments in accordance therewith, it will be apparent to those skilled in the art that various modifications and improvements may be made without departing from the scope and spirit of the invention. Accordingly, it is to be understood that the invention is not to be limited by the specific illustrated embodiments, but only by the scope of the appended claims.

1. A portable sound player for facilitating custom sound therapy for tinnitus management, comprising:
a control unit controlling repeat playback of a recorded sound having a predetermined duration and logging of playback times and volumes;
an audio processing unit operatively coupled to the control unit;
left and right audio outputs operatively coupled to the audio processing unit;
an interface providing data exchanges with an external device; and
a memory unit storing information relating to playback times and volumes.

2. A portable sound player as in claim 1, further comprising a volume control unit configured to adjust the left and right audio outputs to output at different volume levels.

3. A portable sound player as in claim 2, wherein the volume control units comprises separate volume controls for the left and right audio outputs.

4. A portable sound player as in claim 3, wherein the audio processing unit is configured to provide stereo playback at the left and right audio outputs.

5. A portable sound player as in claim 1, further comprising independently buffered audio outputs, each comprising a pair of the left and right audio outputs, thereby allowing simultaneous monitoring of output sound by two persons.

6. A portable sound player as in claim 1, wherein the control unit further controlling logging of playback dates, and the memory unit stores information relating to playback dates.

7. A portable sound player as in claim 6, further comprising an internal date and time of day clock for logging playback dates and times.

8. A portable sound player as in claim 7, wherein the interface provides data exchanges relating to logged playback times and volumes to the external device.

9. A portable sound player as in claim 8, wherein the interface provides download of a sound file from the external device.

10. A portable sound player as in claim 1, wherein the interface receives digital sound data, whereby the control unit controls playback of the received digital sound data via the audio outputs.
